(19) United States
(12) Patent Application Publication
(10) Pub. No.: US 2022/0362554 A1
(43) Pub. Date: Nov. 17, 2022

(54) Supplying a Neurostimulation Signal

(71) Applicant: Robert Steven Gerard Lenzie, Harpenden (GB)

(72) Inventor: Robert Steven Gerard Lenzie, Harpenden (GB)

(21) Appl. No.: 17/740,415

(22) Filed: May 10, 2022

(30) Foreign Application Priority Data
May 12, 2021 (GB) 2106725.1

(51) Int. Cl. A61N 1/36

(52) U.S. Cl. CPC A61N 1/36025; A61N 1/36034

(57) ABSTRACT

Supplying a transcranial alternating current neurostimulation signal to a human brain is shown. A first signal is supplied at a first frequency to said human brain, in combination with a second signal at a second frequency. A third signal at a third frequency is created due to interference between the first signal and the second signal.

### CROSS REFERENCE TO RELATED APPLICATIONS

This application claims priority from United Kingdom Patent Application number 2106725.1, filed on May 12, 2021, the whole contents of which are incorporated herein by reference.

### BACKGROUND OF THE INVENTION

The present invention relates to apparatus for supplying a transcranial alternating current neurostimulation signal to the human brain.

The present invention also relates to a method of supplying a transcranial alternating current neurostimulation signal to a human brain.

Living brains exhibit measurable electrical activity, that can be recorded in the form of an electroencephalogram (EEG). Sometimes brains show abnormal patterns of EEG activity which relate to conditions such as depression, ADHD or sleep disorders. Clinicians are known to supply tACS neurostimulation signals, via scalp electrodes, to the human brain in attempt to correct EEG abnormalities.

It is not currently possible to see the immediate effect that a particular tACS neurostimulation has on an EEG and thereby quickly determine if that neurostimulation is suitable or not for a participant's brain. Under normal conditions, attempting to measure EEG while tACS neurostimulation is being applied to the brain results in the EEG being completely drowned-out by the relative strength of the tACS signal. This is because the magnitude of the EEG is extremely small (10 to 100 micro volts) compared to the magnitude of typical tACS signals (10 to 30 volts). A problem therefore exists in terms of providing an appropriate neurostimulation signal while at the same time ensuring that this signal does not totally mask any output signals of scientific or clinical interest. For this reason, clinicians presently never attempt to view or record EEG while tACS neurostimulation is being given.

### BRIEF SUMMARY OF THE INVENTION

According to a first aspect of the present invention, there is provided an apparatus for supplying a transcranial alternating current neurostimulation signal to a human brain, comprising: a first signal generating device configured to supply a first signal at a first frequency to said human brain; and a second signal generating device configured to supply a second signal at a second frequency to said human brain, wherein: a third signal at a third frequency is created due to interference between said first signal and said second signal.

An embodiment further comprises a receiver for receiving output signals from said human brain in response to the neurostimulation. The receiver may include a low pass filter and an amplifier, such that the first signal and the second signal are applied to the scalp and EEG signals are supplied to the amplifier but the first signal and the second signal are blocked by the low pass filter. The third signal may be supplied to the amplifier via one or more EEG scalp electrodes.

According to a second aspect of the present invention, there is provided a method of supplying a transcranial alternating current neurostimulation signal to a human brain, comprising the steps of: supplying a first signal at a first frequency to said human brain; and supplying a second signal at a second frequency to said human brain, wherein: a third signal at a third frequency is created due to interference between said first signal and said second signal.

In an embodiment, the method further comprises the step of receiving output signals from the human brain in response to the neurostimulation. The receiver may include a low pass filter and an amplifier, such that the first signal and the second signal are applied to the scalp and EEG signals are supplied to the amplifier but said first signal and said second signal are blocked by said low pass filter. The third signal may be supplied to the amplifier via one or more EEG scalp electrodes.

Embodiments of the invention will be described, by way of example only. The detailed embodiments describe the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows an apparatus for applying transcranial magnetic stimulation;

FIG. 2 illustrates a known approach for deploying the apparatus described with reference to FIG. 1;

FIG. 3 illustrates the generation of a third signal in response to receiving two input signals;

FIG. 4 illustrates that the deployment of a thirty-eight-channel electro cap;

FIG. 5 shows a schematic representation of apparatus embodying the present invention; and

FIG. 6 illustrates a method for deploying the apparatus identified in FIG. 5.

### DETAILED DESCRIPTION OF EMBODIMENTS OF THE INVENTION

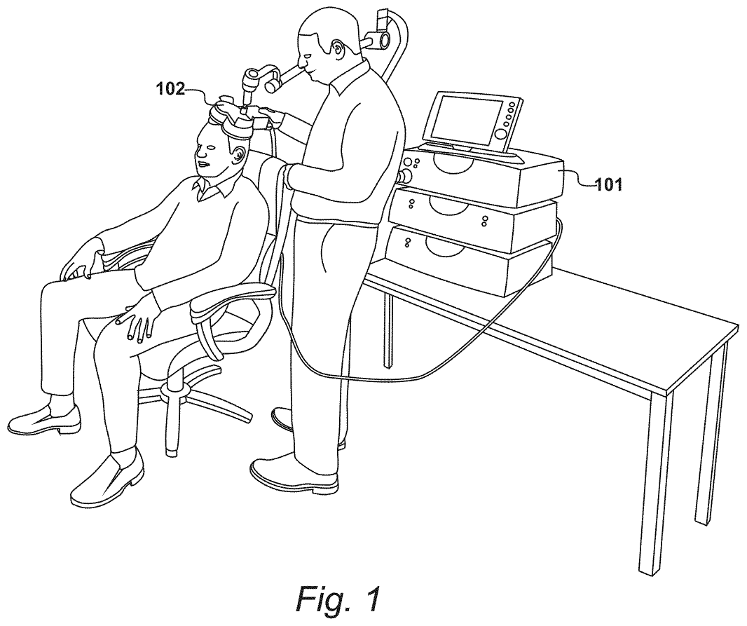

### FIG. 1

An apparatus 101 is shown in FIG. 1 applying transcranial magnetic stimulation, which involves stimulating the brain of a patient with electromagnetic pulses at a frequency of around ten Hz. An electromagnet 102 is often positioned at the top front side of a patient's head and evidence shows that this can be a safe and effective treatment for depression.

Recent experiments have also shown that benefits can be gained from encouraging better communication between regions of the brain—often identified as Brodmann areas, following the work of Korbinian Brodmann. However, for this to be effective, it is necessary to identify appropriate positioning and stimulation frequencies. Thus, after identifying the frequencies that have been found to have an effect, these can be deployed during treatment.

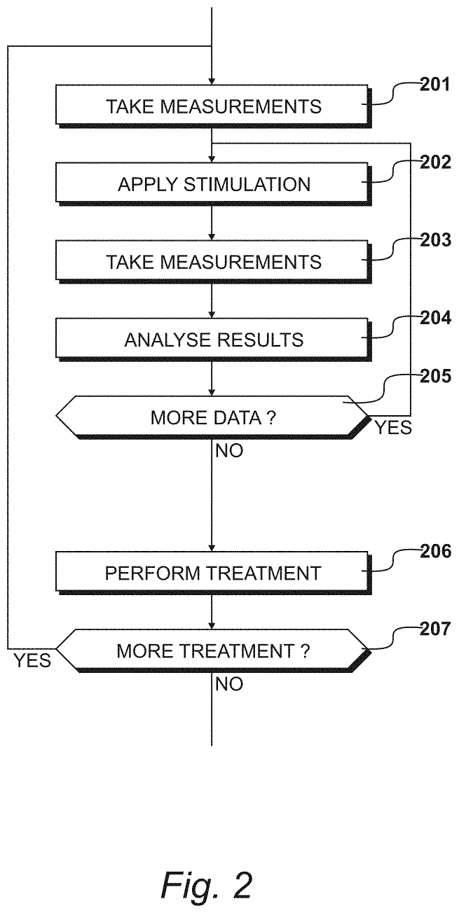

### FIG. 2

A known approach to deploying the apparatus described with reference to FIG. 1 is illustrated in FIG. 2. The treatment step is identified at step 206. Prior to this, investigations are performed to determine how the treatment performed at step 206 may be optimized.

At step 201, initial measurements are taken by means of an EEG in an attempt to identify abnormal patterns of activity.

At step 202, stimulation is applied using a neuro stimulator, typically applying potentials of between ten to thirty volts. It has been recognized that it would be advantageous to take measurements while the stimulation is being applied. However, the magnitude of a typical EEG signal ranges from ten to one-hundred micro volts and as such is totally masked by the stimulation signal. Thus, the best that can be done is to take further measurements at step 203 after the application of stimulation, analyse these results at step 204 and ask a question at step 205 as to whether more data is required. Thus, this process can be repeated until the appropriate data has been received, thereby allowing the treatment to be performed at step 206, followed by a question being asked at step 207 as to whether more treatment is required.

In response to the question asked at step 207 being answered in the affirmative, the whole process is repeated again, starting at step 201.

It has been recognised that a way of improving the efficiency of the TMS procedure, described with reference to FIG. 1, is to tune the stimulation frequency to match the natural resonant frequency of the client's brain.

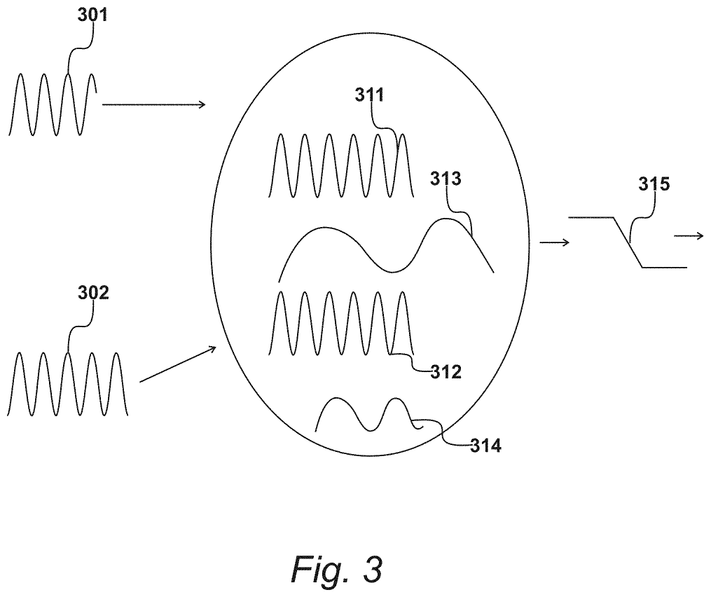

### FIG. 3

Beats are well known in the field of acoustics and are generated when two sounds are created that have slightly different frequencies. The result is perceived as a periodic variation in volume and the beat rate is determined by the difference between the respective frequencies of the generated sounds.

The inventor has realized that a similar approach may be adopted for brain neurostimulation. The literature shows that tACS neurostimulations can have a significant beneficial effect upon a human electroencephalogram (EEG), which can be used for treating a range of neurological and physiological conditions. However, with current systems, it is difficult to determine the immediate effectiveness. In particular, it is very difficult to determine exactly how a given brain responds to a given neurostimulation while the neurostimulation is ongoing.

Potentials measured at the scalp are typically in the range of twenty to one-hundred micro volts, whereas the neurostimulation is typically given at ten to thirty volts. Thus, it is extremely difficult to see brain EEG signals, given that these signals are completely swamped by the size of the stimulating input signals.

The inventor has appreciated that, instead of using two sound sources, it is possible for the brain to be stimulated using two tACS frequencies, whose frequencies of tACS neurostimulation are far outside of the range of the EEG amplifier, which typically has a bandwidth of zero to one-hundred and twenty-eight Hz. Known EEG amplifiers are provided with anti-aliasing filters to remove signals outside of the designated frequency range of operation. Essentially, they are provided with low-pass filters. Thus, by providing a composite interference neurostimulation signal that is far outside the frequency range of these amplifiers, it is possible to deploy frequencies of substantially higher range, typically 1700 Hz to 1800 Hz for example. With a neurostimulation signal being optimally adjusted, the original EEG signal can be seen, overlaid by the neurostimulation signal, emanating from the brain at the required EEG site.

In an example, a neurostimulation frequency of 4 Hz is required. To achieve this, it is possible to set a first tACS source to 1700 Hz and a second tACS source to 1704 Hz. Electrodes are attached to the scalp in the conventional way, selectively receiving the first signal at the first frequency or the second signal at the second frequency. The mix of the two signals induces a 4 Hz wave in the brain, which appears as if a 4 Hz signal had been deployed.

Calibration is required to ensure that the EEG signal remains clearly visible, while at the same time achieving the requisite level of neurostimulation. If the input signal strength is too high, the EEG is completely lost by the neurostimulation signal but if too low, the neurostimulation signal has no visible effect upon the recorded EEG output signals. In an embodiment, it is possible to achieve calibration by the use of a genetic algorithm, with the aim of deploying the minimal amount of neurostimulation in order to obtain the desired effect.

In the example shown in FIG. 3, a first signal 301 has a frequency of eight-hundred-and-fifty-two Hz and a second signal 302 has a frequency of eight-hundred-and-ninety-two Hz. These applied signals propagate through the brain, as illustrated at 311 for the first signal and 312 for the second signal. However, the two applied sine waves interfere to produce a third sine wave 313 having a frequency that is the difference between the frequency of the first signal 311 and the frequency of the second signal 312. In this example, the third signal 313 has a frequency of forty Hz.

The purpose of the third signal 313 is to stimulate the generation of electrical activity within the brain. For the purposes of this example, it is assumed that a fourth signal 314 is produced within the brain as a result of the stimulation created by the third signal 313.

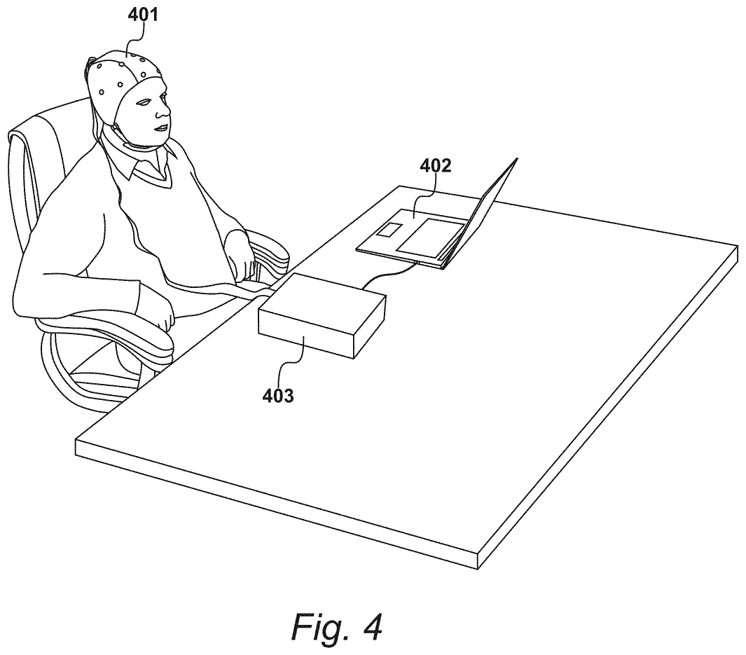

As described with reference to FIG. 4, output signals from the brain are monitored while stimulation is being applied. Output signals are supplied to amplification and monitoring equipment via a low-pass filter 315. As a result of this, the first signal 311 and the second signal 312 cannot pass through the filter 315, given that their frequency is too high. Consequently, on the output side of filter 315, the fourth stimulated signal 314 is seen.

### FIG. 4

In an embodiment, a thirty-eight-channel electro cap 401 is deployed upon a subject, with nineteen of the available channels being used for neurostimulation and the other nineteen channels being arranged to provide an input to a Neurofield Q20 nineteen-channel EEG amplifier; following the international 10/20 standard. In an embodiment, each of the nineteen channels used for neurostimulation are adjacent to each of the standard nineteen channels being routed to the amplifier.

In an embodiment, a genetic algorithm runs on a personal computer 402 and initiates the neurostimulation signals from tACS neurostimulation units 403, from which they are routed to electrodes on the EEG cap, possibly via two digitally controlled 16×16 analog switches. In an embodiment, the control program running on a separate computer system, such as a Raspberry Pi computer, controls the configuration settings and routings of the switches under the control of the genetic algorithm running on the PC. In this way, the analog switch devices can route any one of its tACS neurostimulation units to any one, many or all of the nineteen neurostimulation channels on the thirty-eight channel EEG cap.

Appropriately programmed oscillators produce twelve-volt signals in the 1700 Hz range which are filtered by the anti-aliasing filters of the amplifier. The Q20 amplifier is configured to record signals in a frequency range of zero to one-hundred-and-twenty-eight Hz, with a typical amplitude range of zero to one-hundred microvolts and an EEG resolution of 0.02 microvolts.

The binaural interface pattern between the two alternating current signals, in the range of zero to one-hundred-and-twenty-eight Hz, induces a signal in the brain which may also be seen in real time across one, some or all of the nineteen EEG channels; without the EEG signal being swamped by the noise of the neurostimulation signal. Thus, by adopting this approach, both the EEG and the induced neurostimulation signal can be seen, provided that the amplitude of the neurostimulation signal is adjusted to be just visible at the target electrodes.

The objective of the genetic procedure is to find a neurostimulation location or locations, frequencies and intensities that cause the EEG to change in a predetermined way.

In an embodiment, the genetic procedure starts with a set of ten randomly chosen EEG sites/neurostimulation frequencies/neurostimulation currents. The goal of the genetic procedure is to find a neurostimulation pattern which results in a required change in EEG. Each of these neurostimulations is evaluated and scored. Each neurostimulation is applied to its target site, which may be a single site, many sites or all possible neurostimulation sites and the EEG is recorded as a neurostimulation is applied.

The neurostimulation is automatically calibrated, such that the neurostimulation signal is just visible on at least one of the recorded EEG channels. The neurostimulation that produces the biggest change in EEG as required is carried forward unchanged to the next generation; a process sometimes identified as elitism by those skilled in the art. The remaining nine members of the subsequent generation are created by making random changes to the best neurostimulation from the previous generation. The procedure then continues until there is no significant improvement towards the required change.

Thus, by adopting the approach of this embodiment, it is not necessary to make any assumptions about what configuration of stimulated frequency, intensity and site is likely to work. In an embodiment, the procedure is allowed to find an optimal neurostimulation to create the desired effect.

A possible approach to deploying the apparatus shown in FIG. 4 is to take an initial EEG recording for ten minutes with the subjects' eyes open and then perform this operation again with the subjects' eyes closed. With the eyes closed, the visual cortex starts producing alpha waves. If these are absent, it is likely to indicate that there is some kind of problem present. Thus, this approach allows the brain to be considered in two different states.

The approach then continues by taking a subsequent recording for about thirty minutes during which an electrode is attached to the front of the head with a similar electrode being attached to the back of the head. A range of signals are then applied having frequencies ranging from one Hz to forty Hz. These are played at random, so that the brain does not habituate to an ascending or descending pattern. While the stimulation is being played into the brain, measurements are again taken. As described above, previously it was not possible to record data while stimulation was taking place because the EEG output signals in the range of twenty to fifty micro volts and the stimulation signal is in the range of ten to fifty volts.

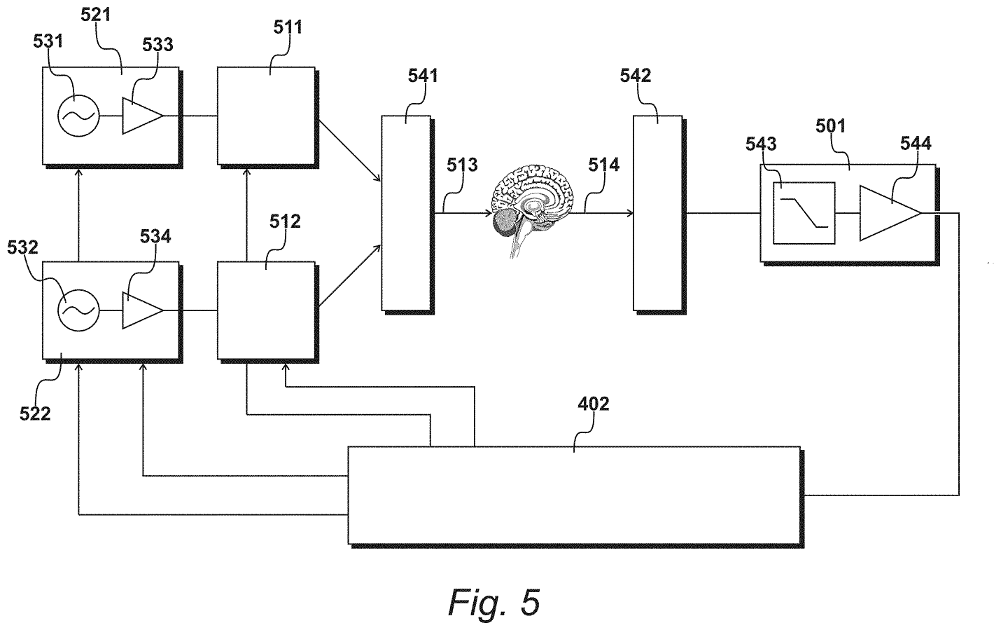

### FIG. 5

As described with reference to FIG. 4, the subject wears a thirty-eight channel EEG cap with nineteen of the channels being used for stimulation and the remaining nineteen channels providing an output to a Q20 EEG amplifier 501.

A first 16×16 analog switch 511 along with a second similar analog switch 512 route alternating stimulation signals to one or more of the nineteen stimulation sites on the thirty-eight channel EEG cap under the control of the computer 402. The nineteen input channels being identified at 513 in FIG. 5, with the nineteen output channels identified at 514.

A first Z3 tDCS/tACS unit 521 is provided with a similar second unit 522; supplying output signals to the first analog switch 511 and to the second analog switch 512 respectively.

The first unit 521 includes a first oscillator 531, with the second unit 522 including a second oscillator 532. The output from the first oscillator 531 is supplied to the analog switch 511 via an amplifier 533. Similarly, the output from the second oscillator 532 is supplied to the second switch 512 via a second amplifier 534.

Outputs from the analog switches 511 and 512 are supplied to the input lines 513 via a first Dsub 25 breakout box 541, with a second similar breakout box 542 supplying signals on lines 514 to the EEG amplifier 501.

As shown in FIG. 5, the signal generating units 521 and 522 along with the analog switches 511 and 512 are controlled by the computer 502. The computer 402 also receives output signals from the EEG amplifier 501.

The EEG amplifier 501 includes, usually as standard, a low-pass anti-aliasing filter 543 and an output amplifier 544. In this way, the twelve-volt signals in the 1700 Hz range produced by units 521 and 522 are blocked by the low-pass anti-aliasing filter 543. The EEG amplifier is designed to record signals in the frequency range of zero to sixty Hz, with an amplitude range of plus or minus one-hundred milli-volts and an EEG resolution of 0.02 microvolts.

Thus, when deployed, a combination of frequencies is played into the brain that are outside of the bandwidth of the EEG amplifier 501. This approach makes use of the amplifier's anti-aliasing filter to cut out frequencies above the desired range and as a consequence of this, the amplifier does not see the stimulation frequencies.

The interference pattern between the two stimulation signals induces a signal in the brain which can be seen in real time across some or all of the nineteen EEG output channels, without the EEG signal being swamped by the amplitude of the stimulation signal. The stimulation units produce a twelve-volt signal and the brain produces a signal in the range of fifty micro volts. Thus, the level of the stimulation signal is substantially greater than the output signal but by adopting this technique, both the EEG and the induced stimulation signal can be clearly seen.

The two high frequencies have a difference between them, therefore the brain experiences frequency differences and follows this stimulation frequency. It is therefore possible to see how the brain responds to specific stimulation frequencies in real time.

Output data received by the computer 402 may be processed locally or transferred for a more in-depth analysis. In an embodiment, commercial software sold under the trademark ‘LORETTA’ may analyse the data by adopting a three shell spherical head model registered to a standardised stereotactic space. This uses the signals received from the surface of the brain to deduce what is happening deep inside the brain.

In an embodiment, the first oscillator 531 is fixed to produce an alternating signal at eight-hundred-and-fifty-two Hz. The frequency of signals produced by the second oscillator 532 is variable, possibly ranging between eight-hundred-and-fifty-two Hz and eight-hundred-and-ninety-two Hz, to give an interference range of between zero to forty Hz. As previously described, these frequencies are applied to the brain at intervals for a fixed period of time in a random order.

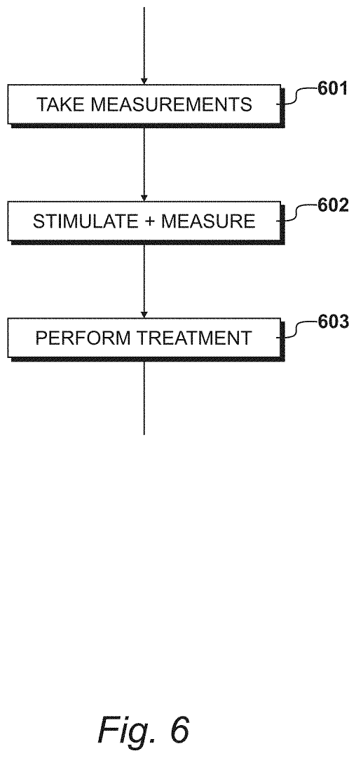

### FIG. 6

By the deployment of aspects of the invention described herein, it is possible to move towards a more simplified working model, compared to that described with reference to FIG. 2. At step 601 initial measurements are taken as described with reference to FIG. 4. At step 602 stimulation and measurement is performed, as described with reference to FIG. 5. Experiments have shown that by being in a position to stimulate and measure at the same time, the usefulness and accuracy of the output data received is substantially enhanced. Thus, it should be possible to move on to performing treatment at step 603 as described with reference to FIG. 1.

This approach is achieved by performing a method of supplying a transcranial alternating current neurostimulation signal to the human brain by supplying a first signal at a first frequency to said human brain and supplying a second signal at a second frequency to the human brain. A third signal is created at a third frequency due to interference between the first signal and the second signal.

1. An apparatus for supplying a transcranial alternating current neurostimulation signal to a human brain, comprising:
a first signal generating device configured to supply a first signal at a first frequency to said human brain; and
a second signal generating device configured to supply a second signal at a second frequency to said human brain, wherein:
a third signal at a third frequency is created due to interference between said first signal and said second signal.

2. The apparatus of claim 1, further comprising a receiver for receiving output signals from said human brain in response to neurostimulation.

3. The apparatus of claim 2, wherein said receiver comprises a low pass filter and an amplifier, such that said first signal and said second signal are applied to a scalp and electroencephalogram (EEG) signals are supplied to said amplifier but said first signal and said second signal are blocked by said low pass filter.

4. The apparatus of claim 3, wherein said third signal is supplied to said amplifier via one or more EEG scalp electrodes.

5. A method of supplying a transcranial alternating current neurostimulation signal to a human brain, comprising the steps of:
supplying a first signal at a first frequency to said human brain; and
supplying a second signal at a second frequency to said human brain, wherein:
a third signal at a third frequency is created due to interference between said first signal and said second signal.

6. The method of claim 5, further comprising the step of receiving output signals from said human brain in response to neurostimulation.

7. The method of claim 6, wherein a receiver that receives said output signals includes a low pass filter and an amplifier, such that said first signal and said second signal are applied to a scalp and electroencephalogram (EEG) signals are supplied to said amplifier but said first signal and said second signal are blocked by said low pass filter.

8. The method of claim 7, wherein said third signal is supplied to said amplifier via one or more EEG scalp electrodes.
